(19) United States
(12) Patent Application Publication
(10) Pub. No.: US 2014/0115730 A1
(43) Pub. Date: Apr. 24, 2014

(54) Apparatuses and Methods for Evaluating and Sorting Pollen and Plants

(71) Applicant: Jason Michael Cope, Ankeny, IA (US)

(72) Inventor: Jason Michael Cope, Ankeny, IA (US)

(73) Assignee: Pioneer Hi-Bred International, Inc., Johnston, IA (US)

(21) Appl. No.: 14/006,605

(22) PCT Filed: Mar. 20, 2012
(86) PCT No.: PCT/US12/29739

§ 371 Date: Jan. 6, 2014

Related U.S. Application Data
(60) Provisional application No. 61/466,350, filed on Mar. 22, 2011.

(51) Int. Cl. A01H 1/04; A01H 1/02; G01N 33/569; C12Q 1/68

(52) U.S. Cl. CPC A01H 1/04; C12Q 1/6895; A01H 1/02; G01N 33/56961
USPC 800/260; 435/6.1; 435/34; 435/7.1; 435/6.11

(57) ABSTRACT

A method for sorting pollen containing genetic elements of interest may include associating a genetic marker with seeds defining the genetic elements of interest, growing plants from the seeds, collecting pollen from the seeds, evaluating the pollen for presence of the genetic markers, and sorting the pollen based on presence or absence of the genetic markers. A method of determining viability of pollen may evaluate the optical density of grains of pollen and compare the optical density to an optical density threshold. Further, a method for identifying plants defining a genetic marker with seeds defining the genetic element of interest, growing plants from the seeds, and evaluating the plants for presence of the genetic marker. Additionally, a method for identifying a genotype of pollen may include evaluating pollen via spectral and/or hyperspectral imaging,

### FIELD OF THE INVENTION

Various embodiments of the present invention relate generally to methods and apparatuses for evaluating and sorting pollen and plants. More specifically, embodiments of the present invention provide methods and apparatuses for evaluating pollen and plants to determine presence or absence of genetic markers or to determine a wavelength pattern associated therewith. Additional methods and apparatuses relate to determining the viability of pollen by evaluating the optical density of the pollen. Pollen may be sorted based thereon.

### BACKGROUND OF THE INVENTION

For a variety of reasons, plant species may be intentionally bred. For example, in some applications plant species are intentionally bred to form hybrid plant species. In some applications, hybrid plants are bred to exhibit various desirable traits. Such traits may include, for example, resistance to heat and drought, resistance to disease and insect damage, improved yield characteristics, and improved agronomic quality. In general, plants may be capable of self-pollination, cross-pollination, or both. Self-pollination describes pollination using pollen from one flower that is transferred to the same or another flower of the same plant. Cross-pollination describes pollination using pollen delivered from a flower of a different plant from a different family or line.

Plants that have been self-pollinated and selected for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of heterogeneous plants that differ genetically and will not be uniform.

Maize (Zea mays L.), often referred to as corn in the United States, may be bred by both self-pollination and cross-pollination techniques. Maize has separate male and female flowers on the same plant. The male flowers are located on the tassel and the female flowers are located on the ear. Natural pollination occurs in maize when wind blows grains of pollen from the tassels to the silks that protrude from the tops of the ears.

The development of a hybrid maize variety in a maize seed production program may involve three steps: (1) the selection of plants from various germplasm pools for initial breeding crosses; (2) self-pollination of the selected plants from the breeding crosses for several generations to produce a series of inbred lines, which, individually breed true and are highly uniform; and (3) crossing a selected inbred line with an unrelated inbred line to produce the hybrid progeny. After a sufficient amount of inbreeding successive filial generations will merely serve to increase seed of the developed inbred. Preferably, an inbred line should comprise homozygous alleles at about 95% or more of its loci.

During the maize inbreeding process, vigor of the line may decrease. Vigor may be restored when two different inbred lines are crossed to produce the hybrid progeny. An important consequence of the homozygosity and homogeneity of the inbred lines is that the hybrid between a defined pair of inbreds may be reproduced indefinitely as long as the homogeneity of the inbred parents is maintained. Once the inbreds that create a superior hybrid have been identified, a continual supply of the hybrid seed can be produced using these inbred parents and the hybrid corn plants can then be generated from this hybrid seed supply.

Accordingly, development and production of maize seed may require pollination at one or more steps. In order to determine that a plant having the desired genetic characteristics is produced by the pollination, genetic trait sampling may be conducted after pollination.

### BRIEF SUMMARY

In one embodiment a method for distinguishing, separating, and sorting grains of pollen containing one or more genetic elements of interest from one or more grains of pollen is provided. The method may comprise associating one or more genetic markers with one or more seeds defining the one or more genetic elements of interest, growing one or more plants from the one or more seeds, and collecting one or more grains of pollen from the one or more plants. Further, the method may include evaluating the one or more grains of pollen for the presence or absence of the one or more genetic markers using an evaluating device, and sorting the one or more grains of pollen defining the one or more genetic elements of interest based on the presence or absence of the one or more genetic markers.

In some embodiments the evaluating device may comprise a single grain flow device. The single grain flow device may be selected from a group consisting of a flow cytometer, a flurometer, a spectroflurometer, and a microfluidic chip. Additionally, evaluating the one or more grains of pollen may comprise conducting at least one of spectral imaging and hyperspectral imaging. In another embodiment, evaluating the one or more grains of pollen may comprise conducting an immunoassay.

In some embodiments the one or more genetic markers may comprise one or more deoxyribonucleic acid-binding proteins. The one or more deoxyribonucleic acid-binding proteins may comprise one or more fluorescent deoxyribonucleic acid-binding proteins. Further, the fluorescent deoxyribonucleic acid-binding proteins may define a plurality of different colors that are respectively associated with different ones of the genetic elements of interest and sorting the one or more grains of pollen may comprise sorting based on the different colors of the one or more fluorescent deoxyribonucleic acid-binding proteins.

Additionally, the method may include germinating one or more additional plants utilizing the one or more grains of pollen defining one or more of the genetic elements of interest. Also, the method may comprise growing a plurality of additional plants by conducting pollen embryogenesis on the one or more grains of pollen defining one or more of the genetic elements of interest. The method may further include dispersing the one or more grains of pollen in a sheath solution prior to evaluating the one or more grains of pollen. The sheath solution may comprise a preservation buffer configured to maintain viability of the one or more grains of pollen. The method may additionally comprise drying the one or more grains of pollen defining one or more of the genetic elements of interest after sorting the pollen, and germinating one or more additional plants utilizing the one or more grains of pollen defining one or more of the genetic elements of interest. Drying the one or more grains of pollen may comprise freeze-drying the one or more grains of pollen defining one or more of the genetic elements of interest.

In some embodiments the one or more genetic elements of interest may comprise a gene. In another embodiment the one or more genetic elements of interest may comprise a quantitative trait locus. Further, associating the one or more genetic markers with the one or more seeds may comprise one or more of transformation and regeneration, traditional breeding, and in situ hybridization of the one or more seeds with deoxyribonucleic acid, ribonucleic acid, or oligonucleotide probes. Additionally, the method may include sorting the pollen based on a time of flight. The method may further include sorting the pollen based on an optical density.

In an additional embodiment a method for determining viability of one or more grains of pollen is provided. The method may comprise evaluating an optical density of the one or more grains of pollen using an evaluating device, comparing the optical density of the one or more grains of pollen to an optical density threshold, and determining viability of the one or more grains of pollen based at least in part on whether the optical density exceeds the optical density threshold. Also, the method may include sorting each of the one or more grains of pollen based at least in part on whether the optical density exceeds the optical density threshold. Further, the method may comprise dispersing the one or more grains of pollen in a sheath solution prior to evaluating the optical density of the one or more grains of pollen.

In an additional embodiment a method for identifying plants defining a genetic element of interest is provided. The method may include associating one or more genetic markers with one or more seeds defining one or more genetic elements of interest, growing one or more plants from the one or more seeds, and evaluating the plants for the presence or absence of the one or more genetic markers using an evaluating device. Evaluating the one or more plants may comprise conducting at least one of spectral imaging and hyperspectral imaging.

A method for identifying a genotype of a grain of pollen is also provided. The method may include collecting a grain of pollen from a plant, evaluating the grain of pollen by conducting at least one of spectral imaging and hyperspectral imaging on the grain of pollen to determine a wavelength pattern, and comparing the wavelength pattern to one or more known wavelength patterns to determine the genotype of the grain of pollen.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates an embodiment of a method for distinguishing, separating, and sorting grains of pollen containing one or more genetic elements of interest from one or more grains of pollen in accordance with an example embodiment of the present invention;

FIG. 2 illustrates a flow cytometer in accordance with an example embodiment of the present invention;

FIG. 3 illustrates a schematic illustration of an evaluating and sorting device of the flow cytometer of FIG. 2 in accordance with an example embodiment of the present invention;

FIG. 4 illustrates a method for identifying a genotype of a grain of pollen in accordance with an example embodiment of the present invention;

FIG. 5 illustrates a method for determining viability of a grain of pollen in accordance with an example embodiment of the present invention;

FIG. 6 illustrates grains of pollen in various states of viability in accordance with an example embodiment of the present invention; and

FIG. 7 illustrates a method for identifying plants defining a genetic element of interest in accordance with an example embodiment of the present invention.

### DETAILED DESCRIPTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

The inventors of the present invention have determined that pollination followed by genetic trait sampling after the pollination may be undesirable. In this regard the inventors have determined that genetic characteristics of the pollen and the plant may be identified prior to pollination. Thereby, for example, pollination may result in a predetermined gamete cross.

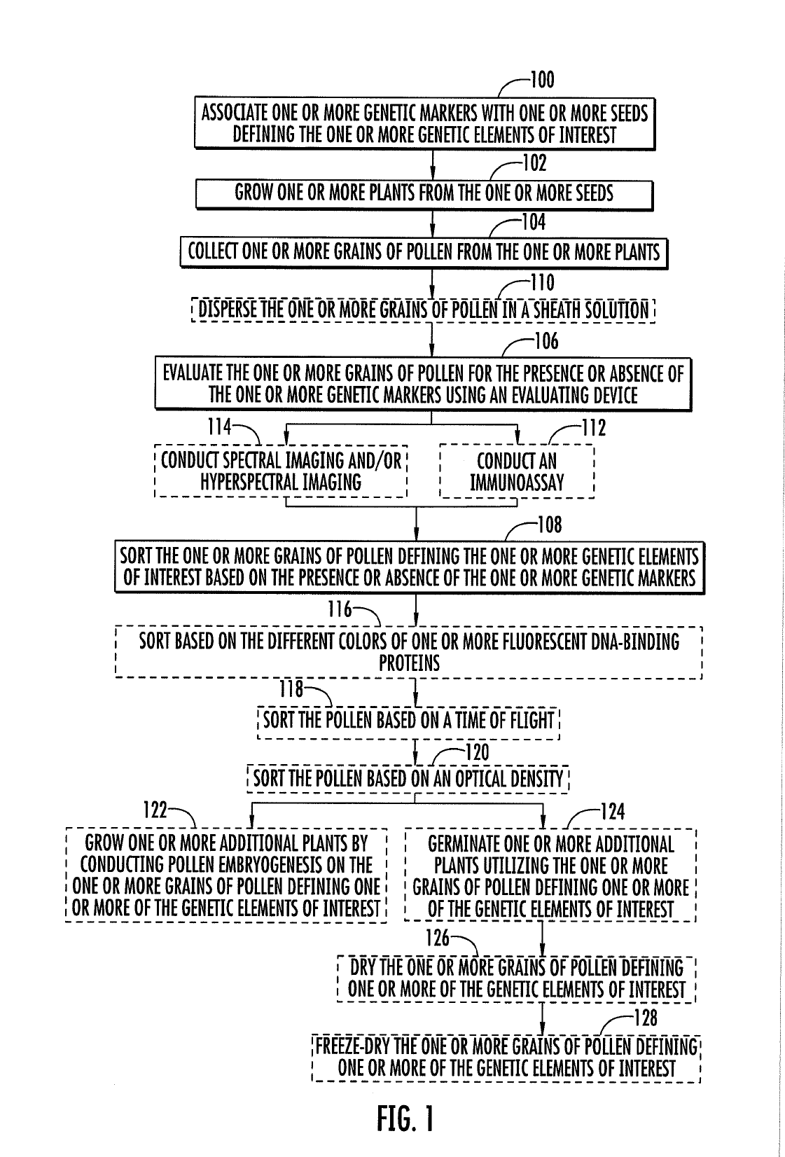

Accordingly, some embodiments of the invention relate to methods for distinguishing, separating, and/or sorting grains of pollen containing a genetic element of interest from one or more grains of pollen. As illustrated in FIG. 1, an embodiment of an example method may include associating one or more genetic markers with one or more seeds defining one or more genetic elements of interest at operation 100. For example, the genetic markers may comprise one or more deoxyribonucleic acid (DNA)-binding proteins. In particular, in one example embodiment that is discussed herein in detail, the DNA-binding proteins may comprise fluorescent DNA-binding proteins. Thus, the fluorescent DNA-binding proteins may define a plurality of different colors that may be respectively associated with different ones of the genetic elements of interest. A genetic element of interest may comprise a gene or a quantitative trait locus (QTL) in various embodiments. For example, certain genes and/or quantitative trait loci may correspond to desirable traits, such as drought resistance, and hence genetic markers may be associated therewith.

A variety of methods and apparatuses may be employed to associate the genetic markers with seeds defining genetic elements of interest at operation 100. For example, associating the genetic markers at operation 100 may comprise transformation and regeneration and/or traditional breeding. Further, associating the genetic markers at operation 100 may include in situ hybridization of the one or more seeds with DNA, ribonucleic acid (RNA), or oligonucleotide probes. Also, various other methods for associating genetic markers with genetic elements may be employed as may be understood by one having skill in the art, such as various other embodiments of genetic sequence insertion.

The method may further comprise growing one or more plants from the one or more seed at operation 102. Additionally, the method may include collecting one or more grains of pollen from the one or more plants at operation 104. Collecting the grains of pollen at operation 104 may involve collecting the grains of pollen with tassel bags in some embodiments, although other methods may be employed such as through use of pollen traps. The method may also include evaluating the one or more grains of pollen for the presence or absence of the one or more genetic markers using an evaluating device at operation 106. Accordingly, grains of pollen which include the genetic element of interest may be identified. Further, the method may include sorting the one or more grains of pollen defining the one or more genetic elements of interest based on the presence or absence of the one or more genetic markers at operation 108. Thereby, grains of pollen that define the one or more of the genetic elements of interest may be separated from grains of pollen that do not define one of the genetic elements of interest.

In some embodiments the above-described method may additionally or alternatively comprise other operations including those operations illustrated in dashed lines in FIG. 1. For example, the method may include dispersing the one or more grains of pollen in a sheath solution at operation 110. As depicted, dispersing the one or more grains of pollen in a sheath solution at operation 110 may be conducted prior to evaluating the one or more grains of pollen at operation 106. The sheath solution may comprise a preservation buffer such as an isotonic buffer in some embodiments, which may be configured to maintain viability of the grains of pollen. Further, as will be discussed below, the sheath solution may function to transport the grains of pollen during one or more of the operations conducted in performing the method.

With regard to evaluating the one or more grains of pollen at operation 106, the operation may comprise conducting an immunoassay at operation 112 and/or conducting at least one of spectral imaging and hyperspectral imaging at operation 114. Thus, for example, the evaluating device may comprise a single grain flow device in some embodiments. By way of further example, the single grain flow device may comprise a flow cytometer, a flurometer, a spectroflurometer, or a microfluidic chip.

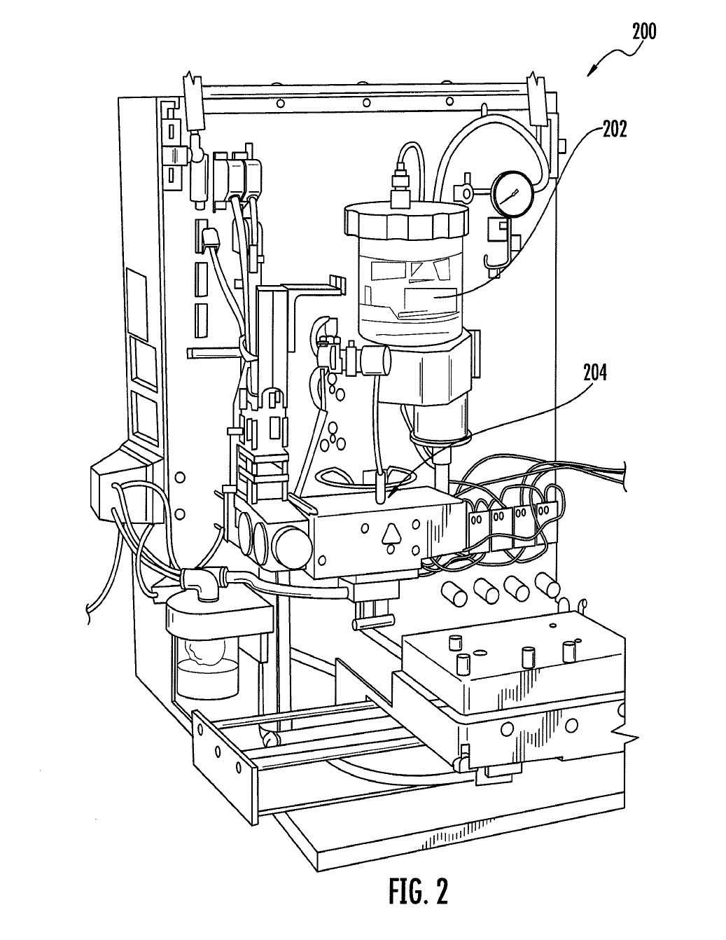

In this regard, FIG. 2 illustrates a flow cytometer 200 which may be employed to perform methods provided herein. In one embodiment the flow cytometer 200 may comprise a flow cytometer sold under the name COPAS by UNION BIOMETRICA, Inc. of Holliston, Mass., although various other embodiments of flow cytometers may be employed. The flow cytometer 200 may comprise a source container 202 which is configured to receive one or more grains of pollen. In particular, the flow cytometer may be configured to receive the one or more grains of pollen dispersed in a sheath solution. The flow cytometer may further comprise an evaluating and sorting device 204 configured to evaluate and sort the grains of pollen.

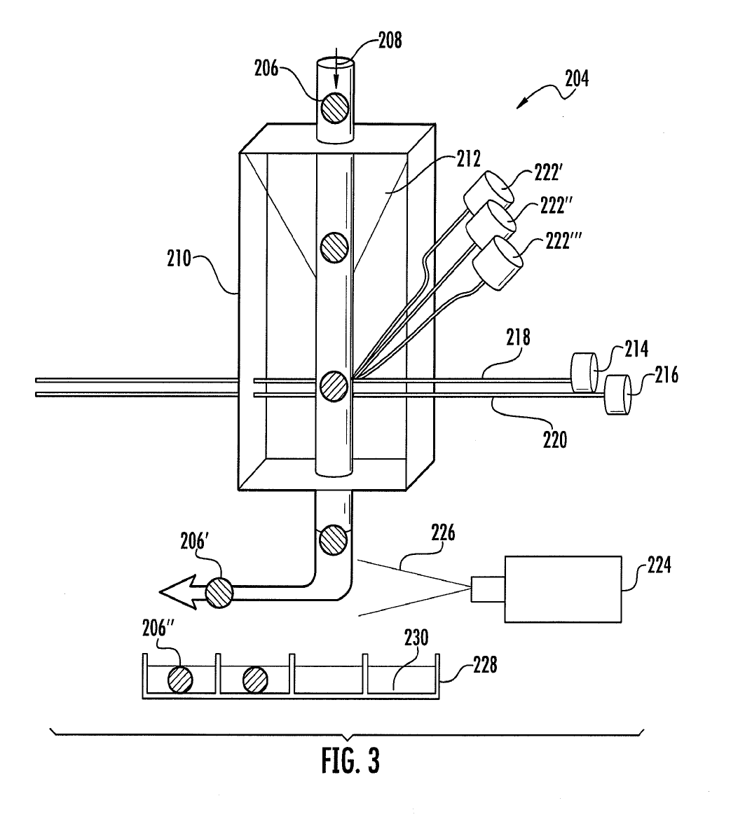

In this regard, FIG. 3 illustrates a schematic illustration of the evaluating and sorting device 204. As depicted the evaluating and sorting device 200 may singulate and direct each grain of pollen 206 along a flow path 208 through a flow cell 210 as caused by a sheath flow 212 of the sheath solution. Accordingly, each grain of pollen 206 may be directed proximate first 214 and second 216 lasers such that first 218 and second 220 laser beams are incident therewith. A plurality of detectors 222 may thereby be configured to detect fluorescence of each grain of pollen 206 as caused by laser excitation. For example, a first detector 222′ may detect red fluorescence, a second detector 222″ may detect green fluorescence, and a third detector 222′″ may detect yellow fluorescence. Thereby, fluorescent DNA-binding proteins may be identified by way of spectral imaging of fluorescence as caused by laser excitation. In this regard, as illustrated in FIG. 1, sorting the one or more grains of pollen at operation 108 may comprise sorting based on the different colors of the one or more fluorescent DNA-binding proteins at operation 116.

Returning to FIG. 3, in some embodiments only the first laser 214 may be employed for evaluating the presence or absence of genetic markers (as indicated by fluorescence). For example, the first laser 214 may be configured to emit the first laser beam 218 in the form of a blue/green laser beam configured to excite fluorescent genetic markers. The second laser 216 may instead emit the second laser beam 220 in the form of a red beam. Thereby the second laser 216 may be employed to determine time of flight of each grain of pollen 206 and optical density (extinction) of each grain of pollen. Time of light may be indicative of the size of each grain of pollen 206. Further, the inventors have determined that optical density may relate to viability of the grains of pollen 206, as will be discussed below. Accordingly, as illustrated in FIG. 1, the method may further comprise sorting the pollen based on a time of flight at operation 118 and/or sorting the pollen based on an optical density at operation 120.

With further regard to sorting the pollen, the evaluating and sorting device 204 of the flow cytometer 200 may comprise a diverter 224. The diverter 224 may be configured to expel a puff of air 226 to divert the grains of pollen 206 when desired. For example, the diverter 224 may divert undesirable grains of pollen 206′ that do not fluoresce or are of the wrong size or optical density to a disposal location. However, the diverter 224 may allow desirable grains of pollen 206″, which may have the genetic marker, to travel to a container 228. The desirable grains of pollen 206″ may be stored in a bulk container or stored in separate compartments 230, as illustrated. In some embodiments the sheath solution may also be directed into the container 228 so as to maintain viability of the desired grains of pollen 206″ after sorting.

With further regard to the method of FIG. 1, the method may additionally comprise growing one or more additional plants by conducting pollen embryogenesis on the one or more grains of pollen defining one or more of the genetic elements of interest at operation 122. Alternatively, or additionally, the method may include germinating one or more additional plants utilizing the one or more grains of pollen defining one or more of the genetic elements of interest at operation 124. As noted above, in some embodiments the grains of pollen 206 defining one or more genetic elements of interest (as may be indicated by one or more genetic markers) may be sorted into a container 228 along with the sheath solution.

Further, the method may comprise drying the one or more grains of pollen defining one or more genetic elements at operation 126. This operation may be conducted after the operation 108 of sorting the pollen, and thereby drying the pollen at operation 126 may involve removing the sheath solution from the grains of pollen. In one embodiment drying the grains of pollen at operation 126 may comprise freeze-drying the one or more grains of pollen defining one or more of the genetic elements of interest at operation 128.

Accordingly, the cytometer 200 or other apparatuses may be employed to evaluate and sort pollen based on the presence or absence of genetic markers, as described above. However, in an alternate embodiment the genotype of a grain of pollen may be identified without associating a genetic marker with seeds. In this regard, the inventors have determined that the genotypes of grains of pollen may be determined through imaging techniques.

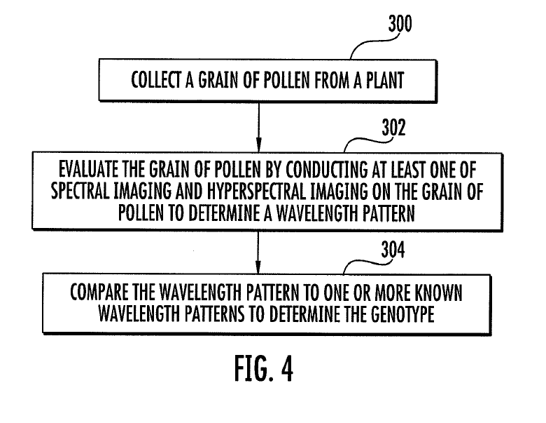

For example, FIG. 4 illustrates an embodiment of a method for identifying a genotype of a grain of pollen. The method may comprise collecting a grain of pollen from a plant at operation 300. Further the method may include evaluating the grain of pollen by conducting at least one of spectral and hyperspectral imaging on the grain of pollen to determine a wavelength pattern at operation 302. Additionally, the method may comprise comparing the wavelength pattern to one or more known wavelength patterns to determine the genotype of the grain of pollen at operation 304. In this regard, each genotype of grains of pollen may define a unique wavelength pattern which may be observed via hyperspectral and/or spectral imaging. Thereby, it may be possible to match the observed wavelength pattern for grains of pollen to known wavelength patterns to determine the genotypes of the grains of pollen. Accordingly, in some embodiments of the invention grains of pollen may be identified without associating genetic markers therewith.

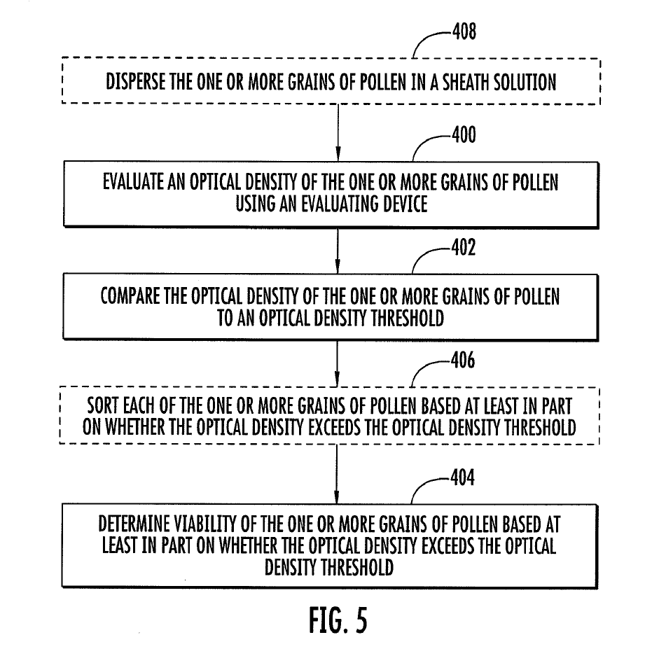

Regardless of the method employed to identify (or evaluate) grains of pollen, additional methods may be employed which may further assist in completing successful pollinations. In this regard, FIG. 5 illustrates a method for determining viability of one or more grains of pollen. As depicted, the method may include evaluating an optical density of the one or more grains of pollen using an evaluating device at operation 400. In some embodiments the evaluating device may comprise the flow cytometer 200 discussed above. In particular, the second laser 216 may be employed to determine the optical density of each grain of pollen 206.

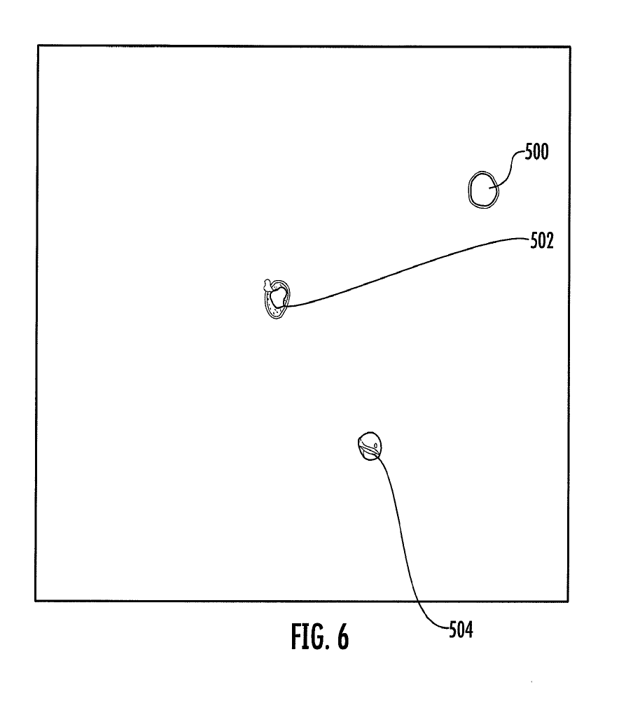

The method may also include comparing the optical density of the one or more grains of pollen to an optical density threshold at operation 402. Additionally, the method may include determining viability of the one or more grains of pollen based at least in part on whether the optical density exceeds the optical density threshold at operation 404. In this regard, the inventors have determined that grains of pollen that have undergone lysis, and hence are less likely to be viable, have a lower optical density than viable grains of pollen. For example, as illustrated in FIG. 6, a viable grain of pollen 500 may have a greater optical density than a partially lysed grain of pollen 502 and a fully lysed grain of pollen 504.

Accordingly, the viability of grains of pollen may be determined based on whether the optical density of each of the grains of pollen exceeds an optical density threshold at operation 404. In one embodiment the optical density threshold may be determined empirically by recording the optical density of a plurality of viable grains of pollen. However, various other embodiments of methods may be employed to select the optical density threshold, as may be understood by one having skill in the art.

In some embodiments the method may additionally or alternatively comprise other operations including those operations illustrated in dashed lines in FIG. 5. For example, the method may include sorting each of the one or more grains of pollen based at least in part on whether the optical density exceeds the optical density threshold at operation 406. As noted above, the evaluating device may comprise the previously discussed cytometer 200. Accordingly, the method may further comprise dispersing the one or more grains of pollen in a sheath solution at operation 408 prior to evaluating the optical density of the one or more grains of pollen at operation 400. As described above, the sheath solution may assist in maintaining pollen viability and may further act as a working medium for moving the grains of pollen.

Thus, the above described methods may provide for identification of the genotype of grains of pollen (see, e.g., FIG. 4) and/or evaluation for the presence or absence of genetic markers associated with genetic elements of interest (see, e.g., FIG. 1). Further, viability of grains of pollen may be determined based on optical density (see, e.g., FIG. 5). These methods may be employed, for example, to prepare pollen for fertilization of plants.

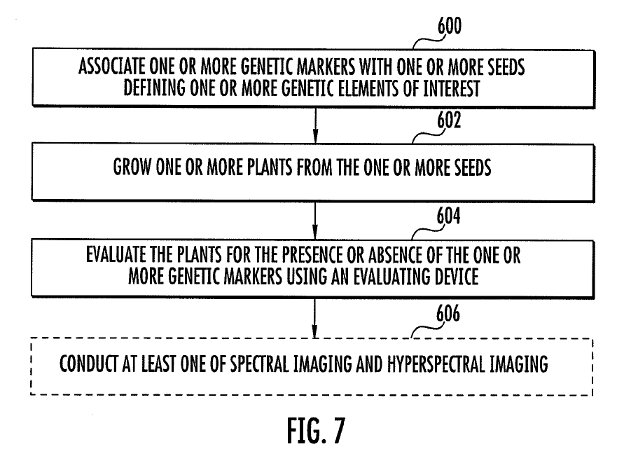

A method for identifying plants defining a genetic element of interest is also provided. As illustrated in FIG. 7, the method may include associating one or more genetic markers with one or more seeds defining one or more genetic elements of interest at operation 600. Further, the method may include growing one or more plants from the one or more seeds at operation 602. Additionally the method may comprise evaluating the plants for the presence or absence of the one or more genetic markers using an evaluating device at operation 604. In some embodiments the method may additionally or alternatively comprise other operations including the operation illustrated in dashed lines in FIG. 7. For example, evaluating the one or more plants at operation 604 may comprise conducting at least one of spectral imaging and hyperspectral imaging at operation 606.

Thereby, for example, plants may be scanned at one or more wavelengths to evaluate the plants for presence or absence of the genetic marker, and hence the genetic elements of interest. In one embodiment plantlet grow outs may be evaluated, and only those plantlets that are determined to have a genetic element of interest (as indicated by one or more genetic markers) may be transplanted and grown. Thus, for example, known female plants may be grown and then pollinated using the known pollen as provided by the methods discussed above. Accordingly, controlled pollination may occur to produced desired plants.

Many modifications and other embodiments of the invention set forth herein will come to mind to one skilled in the art to which these invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for distinguishing, separating, and sorting grains of pollen containing one or more genetic elements of interest from one or more grains of pollen, comprising:
evaluating one or more grains of pollen for the presence or absence of one or more genetic markers using an evaluating device; and
sorting the one or more grains of pollen defining the one or more genetic elements of interest based on the presence or absence of the one or more genetic markers.

2. The method of claim 1, wherein the evaluating device comprises a single grain flow device.

3. The method of claim 2, wherein the single grain flow device is selected from a group consisting of:
a flow cytometer;
a flurometer;
a spectroflurometer; and
a microfluidic chip.

4. The method of claim 1, wherein evaluating the one or more grains of pollen comprises conducting at least one of spectral imaging and hyperspectral imaging.

5. The method of claim 1, wherein evaluating the one or more grains of pollen comprises conducting an immunoassay.

6. The method of claim 1, wherein the one or more genetic markers comprise one or more deoxyribonucleic acid-binding proteins.

7. The method of claim 6, wherein the one or more deoxyribonucleic acid-binding proteins comprise one or more fluorescent deoxyribonucleic acid-binding proteins.

8. The method of claim 7, wherein the fluorescent deoxyribonucleic acid-binding proteins define a plurality of different colors that are respectively associated with different ones of the genetic elements of interest, and
wherein sorting the one or more grains of pollen comprises sorting based on the different colors of the one or more fluorescent deoxyribonucleic acid-binding proteins.

9. The method of claim 1, further comprising germinating one or more additional plants utilizing the one or more grains of pollen defining one or more of the genetic elements of interest.

10. The method of claim 1, further comprising growing a plurality of additional plants by conducting pollen embryogenesis on the one or more grains of pollen defining one or more of the genetic elements of interest.

11. The method of claim 1, further comprising dispersing the one or more grains of pollen in a sheath solution prior to evaluating the one or more grains of pollen.

12. The method of claim 11, wherein the sheath solution comprises a preservation buffer configured to maintain viability of the one or more grains of pollen.

13. The method of claim 11, further comprising drying the one or more grains of pollen defining one or more of the genetic elements of interest after sorting the pollen; and
germinating one or more additional plants utilizing the one or more grains of pollen defining one or more of the genetic elements of interest.

14. The method of claim 13, wherein drying the one or more grains of pollen comprises freeze-drying the one or more grains of pollen defining one or more of the genetic elements of interest.

15. The method of claim 1, wherein the one or more genetic elements of interest comprise a gene.

16. The method of claim 1, wherein the one or more genetic elements of interest comprise a quantitative trait locus.

17. The method of claim 1, wherein associating the one or more genetic markers with the one or more seeds comprises one or more of:
transformation and regeneration;
traditional breeding; and
in situ hybridization of the one or more seeds with deoxyribonucleic acid, ribonucleic acid, or oligonucleotide probes.

18. The method of claim 1, further comprising sorting the pollen based on a time of flight.

19. The method of claim 1, further comprising sorting the pollen based on an optical density.

20. A method for determining viability of one or more grains of pollen, comprising:
evaluating an optical density of the one or more grains of pollen using an evaluating device;
comparing the optical density of the one or more grains of pollen to an optical density threshold; and
determining viability of the one or more grains of pollen based at least in part on whether the optical density exceeds the optical density threshold.

21. The method of claim 20, further comprising sorting each of the one or more grains of pollen based at least in part on whether the optical density exceeds the optical density threshold.

22. The method of claim 20, further comprising dispersing the one or more grains of pollen in a sheath solution prior to evaluating the optical density of the one or more grains of pollen.

23. A method for identifying plants defining a genetic element of interest, comprising:
associating one or more genetic markers with one or more seeds defining one or more genetic elements of interest;
growing one or more plants from the one or more seeds; and
evaluating the plants for the presence or absence of the one or more genetic markers using an evaluating device.

24. The method of claim 23, wherein evaluating the one or more plants comprises conducting at least one of spectral imaging and hyperspectral imaging.

25. A method for identifying a genotype of a grain of pollen, comprising:
collecting a grain of pollen from a plant;
evaluating the grain of pollen by conducting at least one of spectral imaging and hyperspectral imaging on the grain of pollen to determine a wavelength pattern; and
comparing the wavelength pattern to one or more known wavelength patterns to determine the genotype of the grain of pollen.
